(19) United States
(12) Patent Application Publication
(10) Pub. No.: US 2016/0184276 A1
(43) Pub. Date: Jun. 30, 2016

(54) Sphingosine kinase inhibition for the treatment and prevention of obstetrical disorders

(71) Applicant: Sandra Eve Reznik, Larchmont, NY (US)

(72) Inventor: Sandra Eve Reznik, Larchmont, NY (US)

(21) Appl. No.: 14/544,357

(22) Filed: Dec. 30, 2014

(51) Int. Cl. A61K 31/426

(52) U.S. Cl. CPC A61K 31/426

(57) ABSTRACT

The subject invention is directed to the treatment of obstetrical disorders by administration of a sphingosine kinase inhibitor. Spingosine kinase inhibitors are useful in the management or treatment of obstetrical disorders resulting from inflammation such as preterm birth and preterm labor. The treatment may take a variety of forms and is intended for a variety of mammals, such as premature neonates to adult humans.

### CROSS-REFERENCE TO RELATED APPLICATION

This application claims benefit of provisional application No. U.S. 61/964,127 filed on Dec. 23, 2013.

### SUMMARY OF THE INVENTION

The subject invention is directed to the treatment and prevention of obstetrical disorders by administration of an effective amount of a sphingosine kinase (SphK) inhibitor, such as SKI II. Obstetrical disorders include preterm labor, preterm delivery, premature rupture of membranes, prolonged rupture or membranes, prolonged premature rupture of membranes, preterm rupture of membranes, preeclampsia (including mild, severe and superimposed preeclampsia), HELLP syndrome, placental abruption, placenta accreta (including placenta accreta vera, placenta increta and placenta percreta), spontaneous abortion, inevitable abortion, missed abortion, septic abortion and intrauterine fetal demise. The treatment is intended for a variety of mammals, including humans.

Administration of the SphK inhibitor may be performed orally, intravenously, intraperitoneally, subcutaneously, intranasally, transdermally or intramuscularly. SphK inhibitor may be administered alone, or with a carrier such as saline solution, an alcohol, or water. The effective daily amount of the SphK inhibitor is from about 200 μg/kg to about 200 mg/kg of body weight.

### BRIEF DESCRIPTION OF THE DRAWINGS

FIG. 1. SphK inhibition rescues LPS-stimulated timed pregnant mice from preterm birth. Treatment with SKI II significantly reduced the number of mice delivering due to LPS-induced inflammation. In addition, SKI II treatment rescued a significant number of pups carried by LPS-stimulated dams from spontaneous abortion. SKI II significantly improved the rate of pup survival in LPS-treated dams, as seen in the Kaplan-Meier survival curves in Panel C.

FIG. 2. SphK inhibition suppresses expression of placental inflammatory cytokines and endothelin-converting enzyme-1 (ECE-1) in LPS-stimulated timed pregnant mice. Treatment with SKI II significantly suppressed levels of interleukin-1β (3 (Il-1β), interleukin-6 (Il-6), tumor necrosis factor α(Tnfα) and ECE-1 in placentas from timed pregnant mice stimulated with LPS.

FIG. 3. SphK inhibition suppresses cytokine immunoreactivity in the placental labyrinth in LPS-stimulated timed pregnant mice. Treatment with SphK inhibitor SKI II significantly suppressed immunohistochemical staining for Il-1β, Il-6 and Tnfα in placentas from timed pregnant mice stimulated with LPS.

FIG. 4. SphK inhibition suppresses recruitment of inflammatory cells to the placental labyrinth in LPS-stimulated timed pregnant mice. Treatment with SKI II significantly reduced numbers of inflammatory cells in placenta from timed pregnant mice stimulated with lipopolysaccharide.

FIG. 5. Sphingosine kinase (SphK)-endothelin-converting enzyme 1 (Ece1) positive feedback loop. Synthesis of endothelin 1 (Edn1) triggers activation of phospholipase C (PLC), which leads to activation of protein kinase C (PKC) and SphK. Inhibition of SphK with sphingosine kinase inhibitor II suppresses not only several inflammatory cytokines, but also decreases levels of Ece1, which is known to act upstream of SphK. The data support the existence of a positive feedback loop between SphK and the Ece1/Edn1 axis. DAG, diacylglycerol; ET-1, endothelin 1; InsP3, inositol triphosphate; PIP2, phospholipid phosphatidyl inositol 4,5-bisphophate; S1P, sphingosine-1-phosphate.

### DETAILED DESCRIPTION OF THE INVENTION

The subject invention is directed to the treatment of obstetrical disorders by administration of an effective amount of a SphK inhibitor such as SKI II. Obstetrical disorders include preterm labor, preterm delivery, premature rupture of membranes, prolonged rupture of membranes, prolonged premature rupture of membranes, preterm rupture of membranes, preeclampsia (including mild, severe and superimposed preeclampsia), HELLP syndrome, eclampsia, placental abruption, placenta accreta (including placenta accreta vera, placenta increta and placenta percreta), spontaneous abortion, inevitable abortion, missed abortion, septic abortion and intrauterine fetal demise. The treatment is intended for a variety of mammals, including humans.

Administration of the SphK inhibitor may be performed orally, intravenously, intraperitoneally, subcutaneously, intranasally, transdermally or intramuscularly. SphK inhibitor may be administered alone, or with a carrier such as saline solution, an alcohol, or water.

The effective daily amount of the SphK inhibitor is from about 200 μg/kg to about 200 mg/kg of body weight. Preferably, the daily amount is from about 50 mg/kg to about 200 mg/kg, for example about 100 mg/kg body weight of the human being treated (daily). The SphK inhibitor may be administered in any of the methods well known to those skilled in the art. For example, it may be administered orally or intravenously.

Typically, the SphK inhibitor is administered in a pharmaceutically acceptable carrier. Such examples include saline solution, an alcohol, or water. Such carriers are well known in the art, and the specific carriers employed may be varied depending upon factors such as size of the subject being treated, treatment dose, and the like.

Further, the time over which the SphK inhibitor is administered may vary as is well known in the art to achieve the desired results. For example, the SphK inhibitor may be administered intravenously from about 10 minutes to about 1 hour per treatment regimen, 3 times daily, or until the desired daily dosage is fully administered.

Finally, the point in gestation at which the SphK inhibitor is administered may vary to achieve the desired results. For example, the SphK inhibitor may be administered prior to the onset of signs or symptoms of preterm labor to prevent this condition or in the presence of preterm labor to prevent preterm delivery. In another example, the SphK inhibitor may be given before the onset of the signs and symptoms of preeclampsia to prevent this condition or in the presence of preeclampsia to improve this condition and prevent eclampsia.

### Methods

In Vivo Assay. A total of 17 timed pregnant embryonic day (E) 15.5 mice, weighing between 28 and 35 g, were used for in vivo studies. The control group (n=7) was injected intraperitoneally (ip) with 50 mg/kg lipopolysaccharide (LPS) (serotype 026:B6; Sigma) dissolved in 0.5 ml PBS at T=0 and 50 μl PEG 400 at T=1 and 7 h. The treatment group (n=7) was also injected ip with the same dose of LPS and then injected with 50 mg/kg SphK inhibitor (SKI II) dissolved in PEG 400 at T=1 and 7 h. The sham group (n=3) was injected ip with 0.5 ml PBS at T=0 and 50 μl PEG 400 at T=1 and 7 h. After injections at T=7 h, mice were continuously observed for time of delivery and number of pups dropped. Mice that did not deliver were observed until T=24 h. All mice were euthanized by carbon dioxide asphyxiation and necropsied to confirm pregnancy and the number of pups retained in utero was recorded. The retained placentas and uteri were harvested and stored at −80° C. or in 10% neutral buffered formalin at room temperature.

Gel Electrophoresis. Frozen placentas were allowed to thaw on ice. Tissues were homogenized in 0.25 ml ice cold lysis buffer, using a Polytron homogenizer. The samples were homogenized on ice for 2 min at 15 min intervals. This procedure was continued for 2 h and the final homogenates were centrifuged at 10,000×g for 10 min. Volumes of supernatant containing 30-40 mg of protein based on BCA assay, using bovine serum albumin as standard, were diluted in_NuPAGE® LDS sample buffer and the samples were heated at 95° C. for 5 minutes. Bis-Tris gels were used for protein separation. Gel electrophoresis was carried out in an XCell SureLock Mini-Cell apparatus. The gels were allowed to run for 45 minutes with MOPS running buffer at 125 mA and 200V. Proteins were then transferred to a polyvinylidene difluoride (PVDF) membrane, using an XCell II Blot Module. Transfer was carried out for 2 h on ice at a 170 mA current and a constant voltage of 30 V with NuPAGE transfer buffer

Western Blot Analysis. PVDF membranes were washed with Tris-buffered saline containing 0.1% Tween-20 (TBS-T) (Sigma Aldrich, St. Louis, Mo.), pH 7.8 for 5 min and then blocked with 5% skim milk powder (EMD Chemicals) in TBS-T solution for 1 h. The membranes were analyzed for different proteins using their respective primary antibodies, i.e. TNFα, IL-1β, IL-6, IL-10 and ECE-1 diluted in the blocker solution overnight at 4° C. The PVDF membranes were washed with TBS-T three times at intervals of 15 min at room temperature (RT). They were then incubated with a 1:1000 dilution of secondary antibody, anti-rabbit IgG, horseradish peroxidase-linked whole antibody, in blocker solution for 2 h at RT. The membranes were then washed with TBS-T four times at intervals of 15 min at RT. They were then treated with ECL Plus western blotting detection system and the chemiluminescence produced by the chemical reaction was detected by exposure to an autoradiography film. Upon development of the film, the membranes were washed with TBS-T and stored in TBS-T at 4° C. For reprobing, the membranes were treated with stripping buffer for 5 mi at RT. They were then washed with TBS-T three times at 10 min intervals at RT and incubated with primary anti-GAPDH or anti-beta-actin antibody as the gel loading control. The same procedure was repeated as above. The protein content was quantified using Image J software (from NIH). Band density was normalized to housekeeping protein GAPDH or beta-actin. Each placenta subjected to Western blotting analysis came from a different dam. Western blotting experiments were performed at least three times, using at least 3 placentas for each data-point each time.

Histopathological Studies.

Immunohistochemistry (IHC). Formalin fixed paraffin-embedded tissue sections were used for IHC. The sections were deparaffinized by washing in xylene for 3 min. They were then rehydrated by washing in 100% isopropyl alcohol (2 changes), 95% alcohol, 80% alcohol, 70% alcohol and distilled water for 3 min each. The sections were incubated for 5 min with peroxidase block (3% hydrogen peroxide in water) and washed in distilled water for 3 min. Antigen retrieval of the sections was carried out using citrate buffer (8.2 mM sodium citrate, 1.8 mM citric acid-pH 6.0, containing 0.01 per cent Triton X-100) at 95-98° C. for 20 min. The slides were allowed to cool for 20 min and were then washed with Phosphate Buffered Saline (PBS from Sigma) for 3 min. The sections were blocked with 1% bovine serum albumin (BSA) in PBS for 1 h in a humidified chamber. The sections were again washed in PBS for 2 min. Primary antibodies directed against Tnfα, Il-β, Il-6, diluted in 1% BSA, were used for incubation for 1 h in the humidified chamber. The slides were washed in fresh PBS for 3 min. Biotinylated link was applied to the sections for 10 minand the slides were then washed in fresh PBS for 3 min. Streptavidin-HRP was then applied to the sections for 10 min and the slides were again washed in fresh PBS for 3 min. The 3,3′-diaminobenzidine (DAB) substrate-chromogen solution was applied to the sections for 5 min and the slides were washed with distilled water for 3 min. The sections were then immersed in hematoxylin for 2 min and immediately washed in distilled water for 3 min. The slides were wiped and mounted with histochoice mounting media and coverslipped. The sections were observed under a Nikon Ecipse 80i light microscope and pictures were taken with a Nikon Digital Sight camera. Image-Pro Premier DAB analysis software was used for quantification of the results. At least three placentas from three different dams were subjected to immunohistochemical analysis for each condition and each cytokine.

Histology. Placental tissues harvested during necropsy and previously stored in 10% buffered formalin were paraffin embedded, sectioned at 4 mM, mounted on clear glass slides and stained with hematoxylin and eosin (H & E). Sections of the tissues were observed using a Nikon Eclipse 80i light microscope by three blinded observers (V.V., S.S. and S.M.) and graded for extent of inflammatory cell infiltration. The slides were initially scanned at 100× magnification to identify any areas of inflammation. The three most active fields on each slide were then used for analysis at 400×. The number of polymorphonuclear neutrophils (PMNs) were counted in the three fields and the average value was recorded. Based on the average number of neutrophils per 400× field recorded, the slides were assigned grades as follows: 0:0-5 cells, 1:6-50 cells, 2:51-100 and 3:greater than 100. At least three placentas from three different dams were subjected to histologic analysis for each condition and each cytokine. The observers were trained by a practicing pathologist (S.E.R.) before grading began and concordance among the three observers was confirmed with test slides. Images were captured with a Nikon Digital Sight camera.

Statistical Analysis. For in vivo studies, the effect of SKI II on the percent of LPS stimulated mice delivering and on the percent of pups rescued from spontaneous abortion was evaluated with the Chi square and Fisher exact tests, respectively. Differences in rates of PTB and rates of pups delivered over time were evaluated with log-rank (Mantel-Cox) or Gehan-Breslow-Wilcoxon test. The U-test along with Analysis of Variance (ANOVA) and Tukey's multiple comparison test were used to evaluate differences in inflammatory infiltration in histologic sections. Differences in mean expression of cytokine proteins in immunoblots and in immunohistochemistry were determined using ANOVA with Tukey's multiple comparison test.

### Results

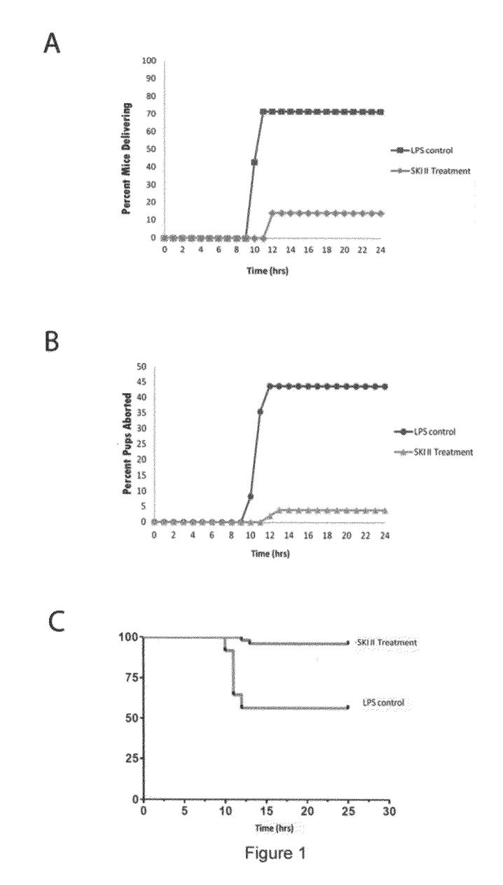

Treatment with 50 mg/kg SKI II significantly reduced the number of mice delivering due to LPS-induced inflammation. In the LPS control group, which received LPS (t=0 h) and PEG 400 (t=1 and 7 h), 5 out of 7 mice (71%) delivered (Table 1, FIG. 1A) by the end of the 24 h experimental period. With SKI II treatment (t=1 and 7 h) following LPS injection, on the other hand, only 1 out of 7 mice (14%) delivered prematurely (Table 1, FIG. 1A) by the end of 24 h, showing a statistically significant effect of SKI II on LPS-induced PTB (P<0.05). None of the three mice in the sham group delivered prematurely, as expected (Table 1, FIG. 1A).

In addition to decreasing the number of mice delivering prematurely, SKI II treatment rescued a significant number of pups from being dropped prematurely and hence spontaneously aborted. In the LPS control group, 21 out of 47 pups (44%) were dropped (Table 1, FIG. 1B) within the 24 h experimental time. In the treatment group, on the other hand, only 2 out of 50 pups (4%) were dropped prematurely in the 24 h time period (P<0.0001) (Table 1, FIG. 1B). In the sham group none of the 20 pups were delivered prematurely, as expected (Table 1). Comparison of the two Kaplan Meier survival curves by log rank test revealed a statistically significant difference between the two groups (P<0.0001) (FIG. 1C). No significant changes in litter size or fetal growth and development were observed throughout the study and no macroscopic congenital anomalies were noted in any of the 97 pups.

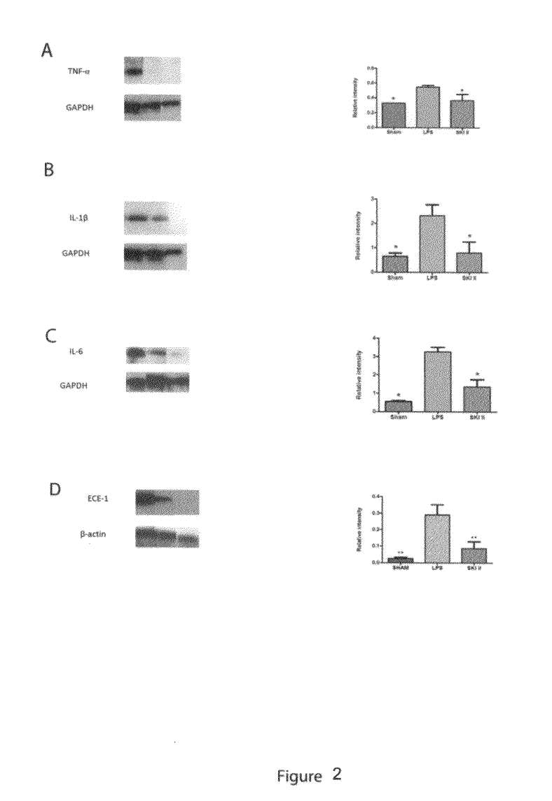

Tnfα, Il-1β and Il6 expression were all significantly increased in placental lysates from LPS-induced mice as compared to shams (P<0.05). Levels of all of these proteins were reduced in the SKI II treated group when compared to the LPS control group (P<0.05) (FIG. 2, A-C). In addition, we found that SKI II clearly suppressed ECE-1 expression (FIG. 2D).

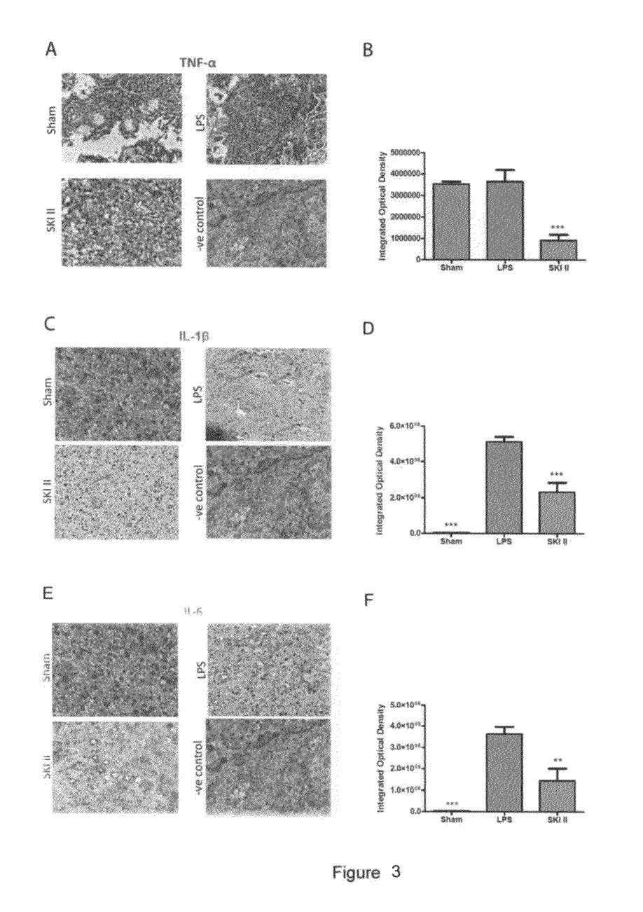

Immunohistochemical analysis was used to determine the localization of cytokines in the placenta and to confirm the results obtained from Western blotting analysis. SKI II significantly reduced mouse placental labyrinthine levels of Tnfa (P<0.0001), Il-1b (P<0.0001) and Il-6 (P<0.01) (FIG. 3).

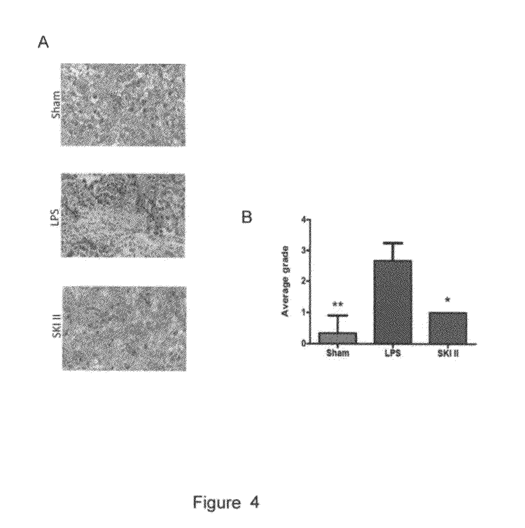

Placental labyrinthine tissue harvested from sham, LPS induced and LPS treated plus SKI II rescued mice showed significant differences in inflammatory cell recruitment. FIG. 4A shows a representative section of placental labyrinth from an LPS-induced mouse with a robust neutrophilic infiltrate when compared to placental labyrinthine tissue from sham mice (P<0.01). With treatment with SKI II, the inflammatory cell infiltrate subsides significantly (P<0.05) (FIGS. 4A and 4B).

### Discussion

This work presents a novel approach to controlling infection-triggered PTB, a clinical challenge accounting for a significant amount of perinatal morbidity and mortality both in the United States and around the globe. Previous studies performed by us and others have implicated the endothelin converting enzyme 1 (ECE-1)/endothelin-1 (ET-1) axis in PTB. However, inhibition of ECE-1 and ET-1 early in gestation results in craniofacial and cardiac abnormalities, revealing its importance for normal embryonic development (Clouthier et al. 2007, Yanagisawa et al. 1998). As ET-1 activates SphK via PKC (Leiber et al. 2007), it was of interest to test whether inhibition of SphK, a downstream mediator in the same molecular pathway as ET-1, would offer a novel approach to controlling PTB without producing teratogenic effects.

The importance of TLR4 in infection-associated PTB has been established by Elovitz et al. (2003), who showed that mice with a mutant form of TLR4 are less, likely to deliver prematurely after LPS induction. Genomic studies in our laboratory show (GEO Access Number GSE 49895, http://www.ncbi.nlm.nih.gov/geo/query/acc.cgi?acc=GSE49895, Sundaram et al. 2013), pointing to Tnfα, Il-1β as Il-6 as important signaling molecules in the LPS-induced inflammatory cascade. In humans, IL-6 in particular is implicated in the pathogenesis of the fetal inflammatory syndrome, a constellation of abnormalities resulting from in utero exposure to inflammation, including periventricular leukomalacia and bronchopulmonary dysplasia (Gomez et al. 2007).

The data presented here suggest that SphK inhibition may prevent PTB triggered by gram negative bacterial infection by suppressing several TLR4-linked proinflammatory cytokines. Although we used a relatively small number of mice in our control and treated groups (n=7 each), the effect of SphK inhibition is so dramatic that our study was adequately powered. The effect of SKI II on dams at risk for PTB, and particularly on pups at risk for spontaneous abortion, was significant (P<0.05 and P<0.001, respectively).

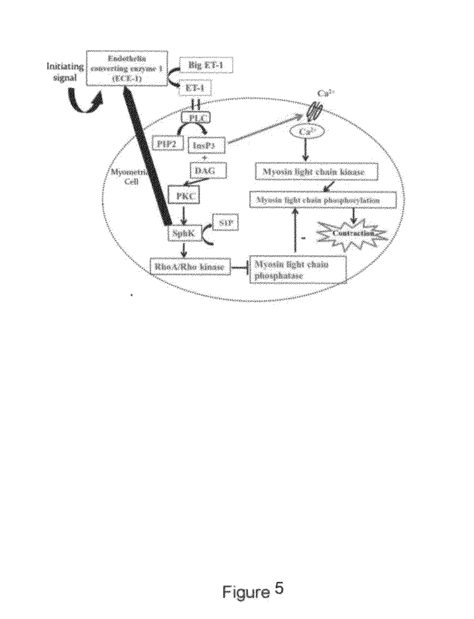

Leiber et al. (2007) have shown that SphK and S1P participate in myometrial contractions in the rat. Their studies indicate that inhibition of SphK causes a reduction in ET-1 mediated myometrial contraction. Based on our finding of decreased placental ECE-1 in LPS stimulated mice treated with SKI II as compared to LPS exposed mice that were not treated, we postulate that the ECE-1/ET-1-PKC-SphK pathway is regulated by positive feedback with increasing activity of SphK in the setting of infection leading to more ET-1 synthesis, leading to greater SphK activity, etc. (FIG. 5). Serrano-Sanchez and colleagues (2008) have shown that cyclooxygenase-2 (COX2) levels correlate with SphK levels in late gestation. We are the first to report, however, that inhibition of SphK controls PTB or any other in vivo model of LPS triggered inflammation based disorder via suppression of cyokine and inflammatory cell infiltration.

### REFERENCES

Clouthier D E, Williams S C, Yanagisawa H, Wieduwilt M, Richardson J A, Yanagisawa M. Develop Bio. 2007, 217:10-24.

Elovitz M A, Wang Z, Chien E K, Rychlik D F, Phillippe M. Am J Path 2003; 163: 2103-2111.

Gomez R, Romero R, Ghezzi F, Yoon B H, Mazor M, Berry S M. Gomez R, Romero R, Ghezzi F, Yoon B H, Mazor M, Berry S M. Leiber D, Banno Y, Tanfin Z. Am J Phy-Cell Phys 2007, 292:C240-C250.

Leiber D, Banno Y, Tanfin Z. Am J Phy-Cell Phys 2007, 292:C240-C250

Serrano-Sanchez M, Tanfin Z, Leiber D. Endocrinol 2008, 149:4669-4679.

Sundaram S, Ashby C R, Pekson R, Sampat V, Sitapara R, Mantell L, Chen C-H, Yen H, Abhichandani K, Munnangi S, Khadtare N, Stephani R A and Reznik S E. Am J Path 183: 422-430 (2013).

Yanagisawa H, Yanagisawa M, Kapur R P, Richardson J A, Williams S C, Clouthier D E, Hammer R E. Develop 1998, 125:825-836.

TABLE 1
SphK inhibition decreases the proportion of lipopolysaccharide
(LPS)-stimulated mice delivering prematurely.

| | | Sphingosine Kinase | |
|---|---|---|---|
| | | Inhibitor II | |
| Group | LPS control | (SKI II) Treatment | Sham |
| LPS (50 mg/kg) Received | YES | YES | NO |
| Number of mice | 7 | 7 | 3 |
| Number of mice | 5 | 1* | 0 |
| delivered | | | |
| Total number of pups | 47 | 50 | 20 |
| Number of pups | 21 | 2**** | 0 |
| spontaneously aborted | | | |

****p < 0.0001
*p < 0.05

What is claimed is:

1. A method of treating an obstetrical disorder with a sphingosine kinase inhibitor.

2. A method of claim 1, wherein the sphingosine kinase inhibitor is administered orally, intravenously, intraperitoneally, subcutaneously, intranasally, transdermally or intramuscularly.

3. A method of claim 1, wherein the sphingosine kinase inhibitor is isolated from a natural source.

4. A method of claim 1, wherein the sphingosine kinase inhibitor is produced by a bioprocess, such as fermentation.

5. A method of claim 1, wherein the sphingosine kinase inhibitor is chemically synthesized.

6. A method of claim 1, wherein the obstetrical disorder is preterm labor.

7. A method of claim 1, wherein the obstetrical disorder is preterm delivery.

8. A method of claim 1, wherein the obstetrical disorder is premature rupture of membranes.

9. A method of claim 1, wherein the obstetrical disorder is prolonged rupture of membranes.

10. A method of claim 1, wherein the obstetrical disorder is prolonged premature rupture of membranes.

11. A method of claim 1, wherein the obstetrical disorder is preterm rupture of membranes.

12. A method of claim 1, wherein the obstetrical disorder is preeclampsia.

13. A method of claim 1, wherein the obstetrical disorder is mild preeclampsia.

14. A method of claim 1, wherein the obstetrical disorder is severe preeclampsia.

15. A method of claim 1, wherein the obstetrical disorder is superimposed preeclampsia.

16. A method of claim 1, wherein the obstetrical disorder is HELLP syndrome.

17. A method of claim 1, wherein the obstetrical disorder is eclampsia.

18. A method of claim 1, wherein the obstetrical disorder is placental abruption.

19. A method of claim 1, wherein the obstetrical disorder is placenta accreta.

20. A method of claim 1, wherein the obstetrical disorder is placenta accreta vera.

21. A method of claim 1, wherein the obstetrical disorder is placenta increta.

22. A method of claim 1, wherein the obstetrical disorder is placenta percreta.

23. A method of claim 1, wherein the obstetrical disorder is spontaneous abortion.

24. A method of claim 1, wherein the obstetrical disorder is inevitable abortion.

25. A method of claim 1, wherein the obstetrical disorder is missed abortion.

26. A method of claim 1, wherein the obstetrical disorder is septic abortion.

27. A method of claim 1, wherein the obstetrical disorder is intrauterine fetal demise.

28. A method of claim 1, wherein the mammal is a human.

29. A method of claim 1, wherein the sphingosine kinase inhibitor is administered with a carrier, such as an alcohol, saline solution or water.

30. A method of claim 1, wherein the effective amount of the sphingosine kinase inhibitor is about 200 μg/kg/day to about 200 mg/kg/day of body weight.
